(19) United States
(12) Patent Application Publication
(10) Pub. No.: US 2026/0014290 A1
(43) Pub. Date: Jan. 15, 2026

(54) SCENT CONTROL APPARATUS

(71) Applicant: Scott A. Elrod, Lake Jackson, TX (US)

(72) Inventor: Scott A. Elrod, Lake Jackson, TX (US)

(21) Appl. No.: 18/771,116

(22) Filed: Jul. 12, 2024

(51) Int. Cl. A61L 9/12; A01M 31/00; A61L 9/04

(52) U.S. Cl. CPC A61L 9/122; A01M 31/00; A61L 9/046; A61L 2209/11; A61L 2209/133; A61L 2209/134; A61L 2209/15; A61L 2209/212

(57) ABSTRACT

A scent control apparatus can include: a housing defining a first aperture and a second aperture; an intake vent and a flow-restricted output vent respectively coinciding at least in part with the first aperture and the second aperture; a fan positioned inside the housing and operable to bring air through the intake vent and flow air directly from the fan and into a chute leading to the flow-restricted output vent; and a cartridge including an oxidant generator coil, the cartridge being removably secured to the housing to position the oxidant generator coil between the fan and the chute.

### FIELD

The described embodiments relate generally to a scent control apparatus, system, and method of scent control.

### BACKGROUND

Animals have an acute sense of smell and are capable of recognizing a human scent (or any other foreign scent that is indicative of possible danger), even at long distances away. To avoid being detected by animals, a person (e.g., a hunter, photographer, researcher, etc.) interested in getting close to an animal will typically attempt to position themselves downwind of the animal or else use commercially available “natural” scents (e.g., animal urine or pine) in attempt to mask or overpower the human scent. However, in reality, such natural scents applied on a human body, clothing, or gear do not accomplish this. Animals will, instead, usually smell both the human and applied natural scents, thereby alerting the animal to the person's presence. Accordingly, there is a need in the art for improved scent control.

The subject matter claimed herein is not limited to embodiments that solve any disadvantages or that operate only in environments such as those described above. Rather, this background is only provided to illustrate one example technology area where some embodiments described herein may be practiced.

### SUMMARY

An aspect of the present disclosure relates to a scent control apparatus. The scent control apparatus can include: a housing defining a first aperture and a second aperture; an intake vent and a flow-restricted output vent respectively coinciding at least in part with the first aperture and the second aperture; a fan positioned inside the housing and operable to bring air through the intake vent and flow air directly from the fan and into a chute leading to the flow-restricted output vent; and a cartridge including an oxidant generator coil, the cartridge being removably secured to the housing to position the oxidant generator coil between the fan and the chute.

In some examples, the scent control apparatus can include a permeable cover positioned over the flow-restricted output vent, the permeable cover configured to induce backpressure in the chute. In particular examples, at least one of the chute, the flow-restricted output vent, or the permeable cover is configured to induce turbulent flow conditions. In one example, a surface of the oxidant generator coil closest to the flow-restricted output vent is configured to be exposed to turbulent airflow.

In one or more examples, at least one of the chute or the flow-restricted output vent is smaller than the intake vent. In specific implementations, the intake vent is elevated relative to the flow-restricted output vent. In some examples, the intake vent and the flow-restricted output vent are both forward-facing. In particular examples, the fan and the oxidant generator coil are aligned relative to each other such that the air exiting the fan proceeds directly in an approximately straight flow path to the oxidant generator coil. In certain examples, the housing defines at least one of an exterior water-shedding feature or an internal moisture evacuation feature. In some examples, the scent control apparatus can include a control interface disposed on a bottom surface of the scent control apparatus.

Another aspect of the present disclosure relates to a scent control system. The scent control system can include a scent control apparatus and a remote device wirelessly coupled to the scent control apparatus (e.g., via a controller of the scent control apparatus). In these or other examples, the scent control apparatus can include: a housing defining housing openings; an upper vent and a lower vent respectively coinciding at least in part with the housing openings and fluidly connected via a fluid flow path, the fluid flow path being constricted in size relative to the upper vent and at a location inside the housing or at the lower vent; a fan configured to intake air through the upper vent and force the air along a direct flow path toward the lower vent; a detachable oxidant generator coil secured to the housing and positioned in the direct flow path; a power supply operable to energize the fan and the detachable oxidant generator coil; and a controller configured to control operation of the scent control apparatus, the controller being communicatively coupled to at least one of the power supply or the fan.

In some examples, the remote device can include a fob. In one or more examples, the remote device can include a client device with a graphical user interface that, in response to user input to the graphical user interface, causes the client device to send a signal to the controller. In particular examples, the graphical user interface includes a visual indicator representative of a status of the detachable oxidant generator coil. In certain examples, the controller can include a processor and a memory device storing computer-executable instructions that, when executed by the processor, cause the processor to transmit a signal to the power supply or the fan. In some examples, the signal causes at least one of: the power supply to change a voltage supplied to the detachable oxidant generator coil; or the fan to change a fan speed. In particular examples, the scent control apparatus is configured to be suspended via a mounting apparatus or lay on top of a support surface.

Yet another aspect of the present disclosure relates to a scent control method performable by a scent control apparatus. The scent control method can include: rotating a fan to bring air through an intake vent; directing the air from the fan and toward a chute aligned with the fan to directly induce contact with at least a rear surface of an energized oxidant generator coil of an oxidant generator insert, wherein turbulent flow conditions within the chute cause additional contact between the air and a front surface of the energized oxidant generator insert opposite the rear surface; forming oxidant based on the air contacting the energized oxidant generator insert; and flowing the oxidant out through an output vent positioned below the intake vent.

In some examples, the oxidant generator insert is detachable from and re-attachable to a housing of the scent control apparatus. In particular examples, the output vent is flow-restricted to: induce the turbulent flow conditions between the energized oxidant generator coil and the output vent; and induce back pressure of airflow into the front surface of the energized oxidant generator coil.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 illustrates an example scent control apparatus and system environment in accordance with one or more examples of the present disclosure;

FIG. 2 illustrates an example environment and use case of a scent control apparatus in accordance with one or more examples of the present disclosure;

FIGS. 3-9 respectively illustrate front perspective, rear perspective, side, front, rear, bottom, and top views of an example scent control apparatus in accordance with one or more examples of the present disclosure;

FIG. 10 illustrates first and second apertures defined by a housing of an example scent control apparatus in accordance with one or more examples of the present disclosure;

FIG. 11 illustrates a close-up view of an example water-shedding feature in accordance with one or more examples of the present disclosure;

FIG. 12 illustrates a bottom perspective view of an example scent control apparatus having a battery pack and oxidant generator cartridge removed in accordance with one or more examples of the present disclosure;

FIG. 13 illustrate an exploded view of an example scent control apparatus in accordance with one or more examples of the present disclosure;

FIGS. 14-18 illustrate various cross-sectional views of an example scent control apparatus in accordance with one or more examples of the present disclosure;

FIG. 19 illustrates a fan and manifold portion of an example scent control apparatus in accordance with one or more examples of the present disclosure;

FIG. 20 illustrates a side cross-sectional view of an example fan, manifold, and chute of a scent control apparatus in accordance with one or more examples of the present disclosure;

FIG. 21 illustrates an example oxidant generator coil cartridge and chute of a scent control apparatus in accordance with one or more examples of the present disclosure;

FIGS. 22-23 illustrate example end portions of an oxidant generator coil cartridge of a scent control apparatus in accordance with one or more examples of the present disclosure; and

FIG. 24 illustrates an example method of scent control performable by a scent control apparatus in accordance with one or more examples of the present disclosure.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The following disclosure relates to a scent control apparatus, a scent control system, and methods of performing scent control (e.g., utilizing a scent control apparatus). In some examples, a scent control apparatus can include a removable oxidant generator. The removable oxidant generator can, in some examples, include a detachable (and re-attachable) cartridge that is securable to the housing of the scent control apparatus. The removable oxidant generator can, therefore, improve ease of use, serviceability, and replaceability according to some examples.

In some examples, the scent control apparatus can include particular fan-chute design that can push airflow in a direct flow path from fan to output vent. The removable oxidant generator can be positioned in alignment with this direct flow path. This shorter, more direct flow path can reduce noise levels and decrease energy draw, in some examples.

Additionally or alternatively, the fan-chute design can impart certain flow characteristics. For example, the chute (and associated flow-restricted output vent) can be tapered or size constricted relative to the intake vent. As another example, the flow-restricted output vent can be partially covered by a permeable cover (e.g., a geometrically arranged front discharge plate overlaying the output vent). In so doing, turbulent flow conditions and/or back pressure can be induced within the chute to cause additional air contact with the removable oxidant generator. This additional air contact can, in turn, increase oxidant generation or oxidant generation efficiency per amount of energy drawn. Additionally or alternatively, internal oxidant levels within the interior volume of the scent control apparatus can be reduced (e.g., as a function of the positional relationship of the chute in close proximity to the output vent).

In one or more examples, the scent control apparatus can include a particular look and feel (e.g., that is conveniently orientable and positionable, even in the dark or in low light conditions). For example, in certain implementations, the scent control apparatus can include an intake vent and the output vent that are both forward facing. In some examples, the intake vent is positioned above the output vent to reduce perceived noise levels and/or limit (or prevent) incidental intake of any oxidant. Additionally or alternatively, the scent control apparatus can be sleek, profiled, or contoured to provide certain moisture-shedding attributes. In some examples, the scent control apparatus can include specific moisture-shedding features (exterior and/or interior) to help reduce or eliminate unintended, premature oxidant reaction with moisture and/or to help prevent moisture contact with the removable oxidant generator.

In some examples, the scent control apparatus can be readily controlled via one or more devices (e.g., a client device or fob) and/or through an ergonomic (and multi-purpose) onboard control interface. The scent control apparatus can be mounted in a variety of configurations via a mounting apparatus (or else operated in an unmounted configuration). The scent control apparatus can also include a power supply and, for rechargeable power supplies, an onboard charging functionality via a charge port. In some examples, the power supply is sized and shaped in a configuration that can provide increased balance and stability for the scent control apparatus.

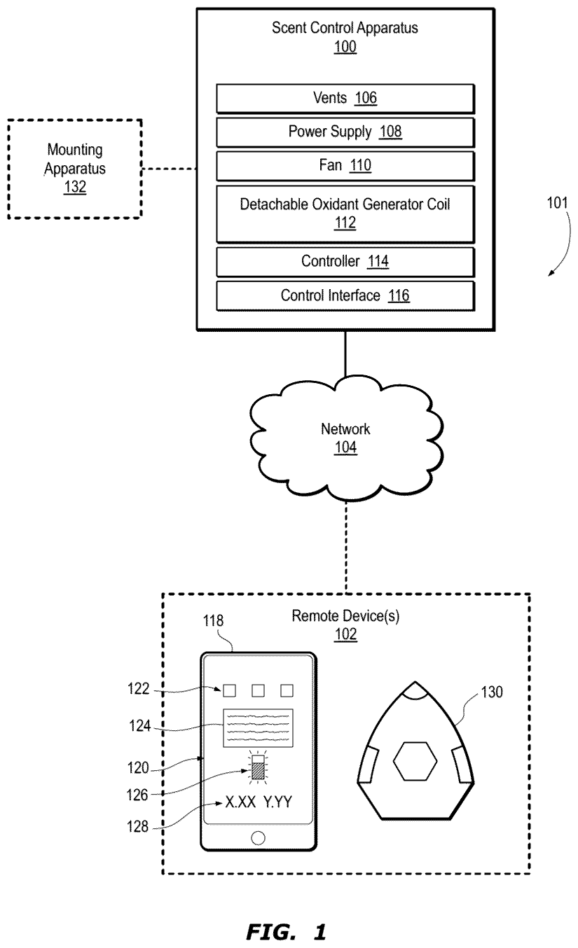

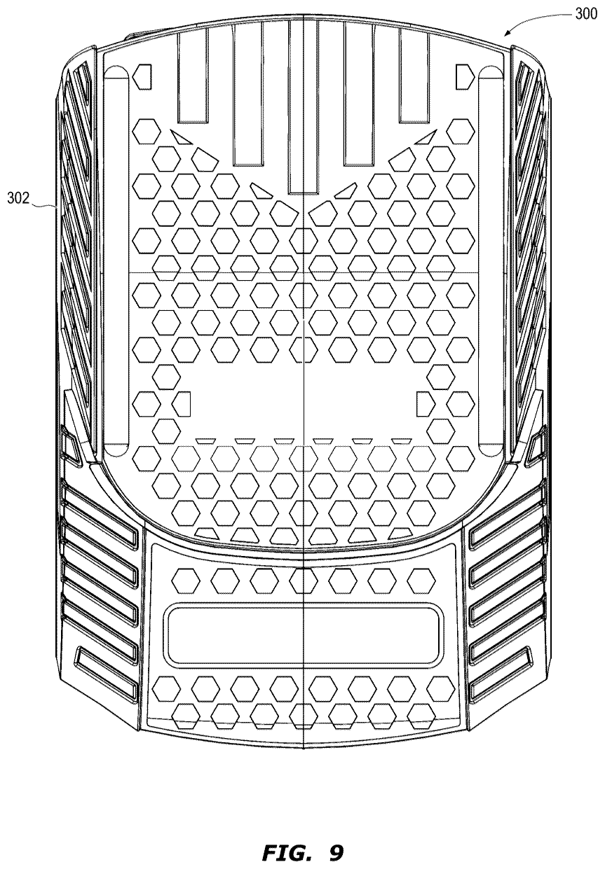

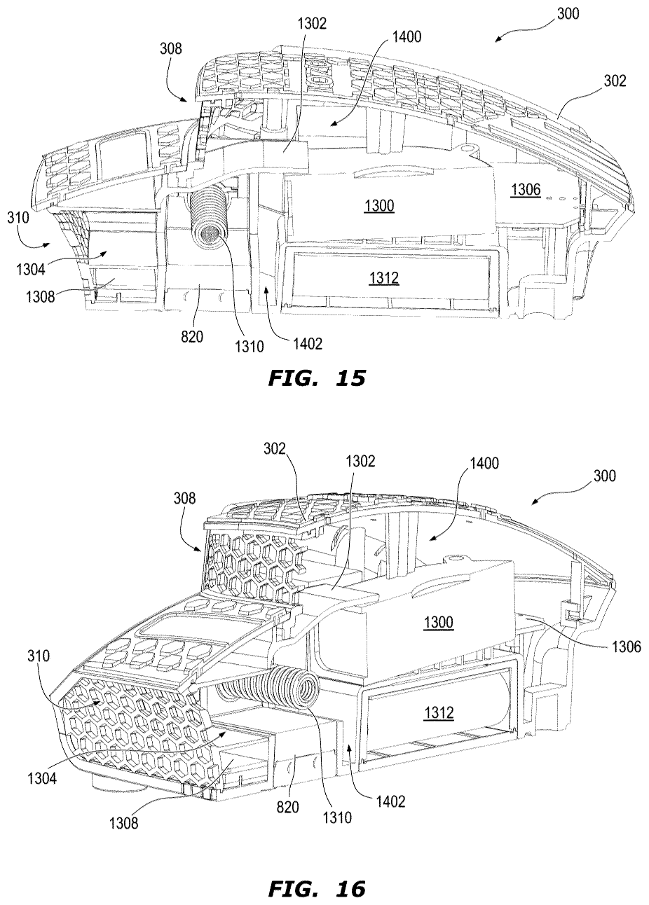

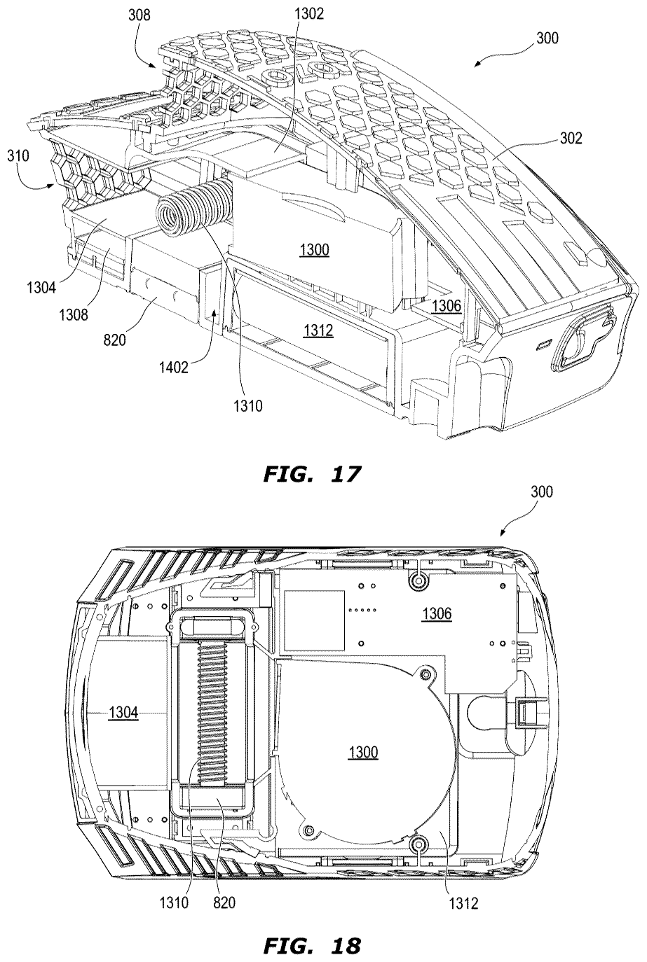

These and other embodiments are discussed below with reference to FIGS. 1-24. However, those skilled in the art will readily appreciate that the detailed description given herein with respect to these figures is for explanatory purposes only and should not be construed as limiting. Furthermore, as used herein, a system, a method, an article, a component, a feature, or a sub-feature including at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1 illustrates an example scent control apparatus 100 and system environment 101 in accordance with one or more examples of the present disclosure. As shown, the system environment 101 can include the scent control apparatus 100 and, optionally, at least one of remote device(s) 102 and/or a mounting apparatus 132. Each is discussed in turn below.

The scent control apparatus 100 can include various structures, components, and arrangements thereof to emit one or more oxidants generated from ambient air. Oxidants can include one or more of ozone, diatomic oxygen, diatomic halogens, peroxides, hydroxyls, radicals of any of the foregoing or components thereof, metastable oxygen, negatively charged metal oxides, encapsulated ozone, activated ozone, peracetic acid, chlorine dioxide, thixotropic gels, singlet oxygen, hypochlorite, or chlorite. The scent control apparatus 100 can, in some examples, include an activated water generator, a peroxide ion generator, and/or a radical generator, such as an electrolytic device for carrying out electrolysis of one or more of water or a peroxide. In particular examples, the scent control apparatus 100 can include a fluid oxidant storage and a mist sprayer operably coupled thereto to spray a mist (e.g., droplets or micro droplets) of fluid oxidant.

Oxidants, including ozone and derivatives thereof (e.g., singlet oxygen, diatomic oxygen, atomic oxygen, metastable oxygen, or activated oxygen), may be particularly suitable for controlling scents. The term “controlling scents” refers to breaking down or reacting scent molecules or scent molecule sources. Thus, unlike a masking aroma, an oxidant as disclosed herein can chemically alter, bind to, or substantially neutralize a scent molecule—thereby effectively eliminating scent molecules and forming unrecognizable derivatives or reactants.

In some examples, the scent control apparatus 100 can dynamically generate oxidant based on local conditions (e.g., wind, temperature, pressure, elevation, humidity, precipitation, sunrise/sunset times, weather forecast, latitude and longitude, etc.). For example, the scent control apparatus 100 can increase oxidant output in response to an increase in wind speed (e.g., increase the oxidant output by at least 10%, such as 10% to 30%, 20% to 30%, about 50%, between 50% and 100%, or greater than 100%) to counter dilution of the oxidant.

Similarly, in some examples, the scent control apparatus 100 can dynamically (e.g., in real time or near-real time, according to GPS data, according to onboard sensor data, according to sensor data of the remote device(s) 102, etc.) adjust inputs based on local conditions. For example, at higher elevations, the amount of available oxygen to convert to an oxidant can be less than an amount of oxygen available at lower elevations. Thus, to maintain a substantially consistent flow and/or concentration of oxidant at higher elevations (for instance), the scent control apparatus 100 can increase a fan speed of the fan 110 to bring in more air into the scent control apparatus 100 and/or increase a voltage applied to the detachable oxidant generator coil 112. In specific implementations, the scent control apparatus 100 can utilize sensors (including non-oxygen sensing sensors like pressure sensors, temperature sensors, position sensors, etc.) that can detect local conditions indicative of an actual, real-time amount of available oxygen in the ambient air. In this way, the scent control apparatus 100 can be independent of (i.e., not reliant upon) oxygen sensors—which, of themselves, can be subject to various inaccuracies and technical difficulties.

In one or more examples, the scent control apparatus 100 can include a portable (e.g., carryable, packable, movable, or stowable) scent control apparatus, a mountable scent control apparatus, a fixed or permanent scent control apparatus, etc. The scent control apparatus 100 can, in particular examples, be utilized as a tool for hunters, photographers, researchers, etc. to cleanse the air of human scent between a user and an animal. However, the scent control apparatus 100 is not so limited. Indeed, the scent control apparatus 100 can be implemented and/or tailored for use in a wide variety of applications, including industrial applications, sanitization applications, vehicular applications, mold and/or allergen reduction applications, deodorizer applications, etc. The scent control apparatus 100 can additionally or alternatively be implemented in homes, medical offices, hotel rooms, lockers, vehicles, etc. In some examples, the scent control apparatus 100 can also be used on clothing, furniture, fabrics, carpet, and the like.

In these or other examples, the scent control apparatus 100 can include vents 106 (e.g., apertures, ducts, orifices, openings, etc.). The vents 106 can include an intake vent through which air (e.g., ambient environment air) passes into the scent control apparatus 100. Additionally, the vents 106 can include an output vent through which oxidant is emitted from the scent control apparatus 100.

As will be described in more detail below, the vents 106 can include a variety of structural configurations and positionings. In particular examples, the vents 106 can include an upper vent and a lower vent corresponding to the intake vent and the output vent, respectively. That is, in some examples, the intake vent can be elevated (i.e., raised relative to or positioned at least partially above) the output vent. In this manner, when the scent control apparatus 100 is mounted above a user, the intake vent can be positioned farther away from the user and/or oriented in a direction that can reduce perceived noise levels. Additionally or alternatively, the vents 106 can include first and second vents that are forward-facing (e.g., an orientation to intake air and output oxidant in a substantially frontward direction). Alternatively, in some examples, the vents 106 can have differing orientations (e.g., a side or rear-facing intake vent and a forward-facing output vent).

In one or more examples, the vents 106 can also have a variety of shapes, sizes, and/or permeable covers (e.g., pass-through grates, grilles, barriers, screens, etc.). The various structural configurations of the vents 106 can induce certain fluid flow characteristics (e.g., pressure, velocity, turbulence) of air and/or oxidant inside the scent control apparatus 100 or upon exiting the scent control apparatus 100. For instance, a permeable cover can be disposed over the output vent and/or the output vent can be constricted in size (e.g., have a smaller perimeter) relative to the intake vent. In so doing, backpressure can increase and/or turbulent fluid flow inside the scent control apparatus 100 can be induced.

The scent control apparatus 100 can include a power supply 108. As used herein, the term “power supply” refers to any power source that can provide power to one or more components of the scent control apparatus 100 (e.g., for powering a fan motor and/or energizing a detachable oxidant generator coil). For example, a power supply can include fuel cells, battery cells, generators, alternators, solar power converters, motion-based converters (e.g., that convert vibrations or oscillations into power), etc. In particular implementations, a power supply can convert alternating current to direct current (or vice-versa) for powering or charging/recharging components of the scent control apparatus 100. Some particular examples of a power supply can include a switched mode power supply, an uninterruptible power supply, an alternating current power supply, a direct current power supply, a regulated power supply, a programmable power supply, a computer power supply, and a linear power supply. In some examples, a power supply includes a rechargeable battery pack (e.g., including one or more lithium-ion cells).

The scent control apparatus 100 can include a fan 110. The fan 110 can include structural component(s) for pulling in ambient air through the intake vent (of the vents 106) and/or directing intake air toward a detachable oxidant generator coil 112 and pushing formed oxidant out through the output vent (of the vents 106). The fan 110 can include one or more of a variety of different types of fans. For example, the fan 110 can include an axial fan, centrifugal fan, blower, vaned fan, propeller fan, mixed-flow cooling fan, tubular airflow fan, an impeller, etc. The fan 110 can include a cage fan according to any of the foregoing types of fan. In particular examples, the fan 110 can push air perpendicular (or substantially perpendicular) to its axis of rotation (e.g., such that airflow can move in a direct path from the fan 110 to the detachable oxidant generator coil 112, as will be discussed below).

The scent control apparatus 100 can include the detachable oxidant generator coil 112. The detachable oxidant generator coil 112 can generally refer to a corona discharge generator, a corona discharge plate, an ultraviolet ozone generator, an electrolytic ozone generator, or any other type of ozone generator. The detachable oxidant generator coil 112 can include a variety of different shapes and sizes. Indeed, the detachable oxidant generator coil 112 is not limited to a cylindrical shape or coil shape. In some examples, the detachable oxidant generator coil 112 includes an ionizer (e.g., a negative ion generator) or electrostatic precipitator. Additionally or alternatively, the detachable oxidant generator coil 112 can provide a source of peroxides or derivatives thereof (e.g., hydroperoxides, hydroxyl radicals, or peroxide radicals). For example, a catalytic ionizer may provide oxidants. Catalytic ionization of air by ultraviolet light may produce a mixture of hydroxyl ions, hydroxyl radicals and hydrogen peroxide ions (as well as ozone).

In particular examples, the detachable oxidant generator coil 112 is detachable. As referred to herein, the term “detachable” can refer to various insert-like attributes of the detachable oxidant generator coil 112, including replaceable, interchangeable, swappable, retractable, insertable, removable, etc. In particular examples, the detachable oxidant generator coil 112 can be conveniently removed for cleaning or refurbishing. Additionally or alternatively, the detachable oxidant generator coil 112 can be substituted with a replacement oxidant generator coil 112.

In these or other examples, the detachable oxidant generator coil 112 can be attached to (e.g., removably attached to or integrally formed within) a cartridge. The cartridge of the detachable oxidant generator coil 112 can include a housing, shell, holder, retaining mechanism, etc. for the detachable oxidant generator coil 112. Thus, at the time of replacement or substitution, the detachable oxidant generator coil 112 can be switched out along with the cartridge (e.g., insert a whole different cartridge and different oxidant generator coil). Alternatively, the detachable oxidant generator coil 112 can be removed from the cartridge itself such that a new coil is attached to the old cartridge, and both are re-inserted into the scent control apparatus 100. Regardless of the replacement method, the detachable oxidant generator coil 112 can advantageously impart improved convenience and ease of self-maintenance over prior systems and methods in the art that implement ozone generating element(s) not designed for removal.

In one or more examples, the scent control apparatus 100 includes a controller 114. The controller 114 can control operation of the scent control apparatus 100. To do so, the controller 114 can be communicatively coupled to the various components of the scent control apparatus 100. In some examples, the controller 114 is communicatively coupled to the components of the scent control apparatus 100 via the network 104 (described below). Additionally or alternatively, the controller 114 is coupled to the components of the scent control apparatus 100 via at least one of electrical and/or data connections for power and/or data throughput.

In certain implementations, the controller 114 can include at least one of a processor or a memory device. The processor can include a system on chip, integrated circuit, driver, microcontroller, application processor, crossover processor, etc. The processor can also include circuitry and associated circuit boards, connectors, that electrically couple components together, or other suitable electronic components (e.g., resistors, capacitors, inductors, potentiometers, transformers, diodes, transistors, etc.). In these or other examples, the processor can execute computer-executable instructions received from the memory device, another component of the scent control apparatus 100, or the remote device(s) 102. Additionally or alternatively, the processor can receive a signal from one or more components (e.g., the control interface 116 or the remote device(s) 102).

In response to a received signal and/or executing the computer-executable instructions, the processor can transmit a signal to one or more components. For example, the processor can transmit a signal to the power supply 108 or the fan 110. In such an example, the transmitted signal to the power supply 108 can cause the power supply 108 to change a voltage supplied to the detachable oxidant generator coil 112 (thereby increasing or decreasing the amount of oxidant generated). The transmitted signal to the fan 110 can similarly, for instance, cause the fan 110 to change a fan speed (thereby increasing or decreasing the amount of oxygen intake).

The memory device can include one or more memory devices (e.g., individual nonvolatile memory, processor-embedded nonvolatile memory, random access memory, memory integrated circuits, DRAM chips, stacked memory modules, storage devices, memory partitions, etc.). The memory device can store computer-executable instructions, including those described above.

The scent control apparatus 100 can additionally include a control interface 116 for providing user input to the scent control apparatus 100 and/or identifying various statuses or warnings of the scent control apparatus 100 (e.g., battery life, oxidant generator coil life, temperature notifications, etc.). The control interface 116 can include one or more of buttons, switches, displays, indicators, and the like. In particular examples, the control interface 116 can include such user input elements for switching between or initiating various operating modes, powering the device on and off, pairing the scent control apparatus 100 with a remote device, etc.

The remote device(s) 102 can include at least one of a client device 118 or a fob 130. Both of the client device 118 and the fob 130 can be used to remotely (e.g., wirelessly) control or engage with the scent control apparatus 100. Such remote access to the scent control apparatus 100 can, in some examples, be advantageous when sound and/or movement to manipulate the scent control apparatus 100 would otherwise be difficult or undesirable (particularly when in close proximity to an animal with keen senses).

The client device 118 can include a variety of electronic devices. In some examples, the client device 118 can include a smart phone device. However, the client device 118 can include one or more of a variety of different types of computing devices (or electronic devices), such as notebook computers, desktop computers, tablets, wearables, watches, head-mountable devices (e.g., smart glasses, augmented reality and/or mixed reality headsets), audio devices (e.g., ear buds, headphones, ear muffs), servers, similar devices, and combinations thereof. In at least one implementation, the client device 118 can include a human-computer interface (e.g., a brain-computer interface implanted in a user's brain tissue) that can operate the scent control apparatus 100 via physiological signals, neural signals, electrochemical signals, etc. that are generated within the human body. Indeed, the client device 118 is shown merely as an example device with which aspects of the present disclosure are illustrated for convenience in providing an explanation and should not be viewed as limited to a tablet computing device or smart phone.

As shown, the client device 118 can, in certain examples, include a graphical user interface 120. The graphical user interface 120 can include a variety of custom prompts, colors, indicators, settings, operating modes, and/or notifications in relation to the scent control apparatus 100. The graphical user interface 120 can be dynamically generated via pixels that can populate with a respective pixel color according to computer-executable instructions-all for user viewing through a display (e.g., a glass display cover). More generally, the graphical user interface 120 can include any visual element or user interface component visually displayed on the client device 118 to remotely engage with (e.g., operate, control, identify statuses, monitor, update, etc.) the scent control apparatus 100. As some specific examples, the graphical user interface 120 can include buttons 122. The buttons 122, in response to user input (e.g., a button press, tap, or hold), can cause the client device 118 to transmit a signal to the scent control apparatus 100. In some examples, the buttons 122 can include a mode button (e.g., “Boost,” “Hyperboost,” “Locker,” “Driwash,” “Standard”), power button, information button, device pair button, etc. In particular examples, the graphical user interface 120 can include a notification 124. The notification 124 can include a prompt, warning (e.g., regarding the scent control apparatus 100, inclement weather, etc.), recommendation (e.g., which direction to point the scent control apparatus 100 based on the local conditions), or notice (e.g., a software update notice).

In one or more examples, the graphical user interface 120 can include an indicator 126. The indicator 126 can include a visual indicator (e.g., a graphic, symbol, icon, etc.) representative of a status of the detachable oxidant generator coil 112. For example, the indicator 126 can include a real time (or near real time) graphical representation of how many oxidant-generating hours remains for the detachable oxidant generator coil 112 before replacement or maintenance is due. The client device 118, in some examples, can generate the indicator 126 by utilizing a timer and measuring applied voltage to determine, in real time, an amount of operation time and “experienced” voltage to approximate a relative lifetime status against one or more data values for actual or simulated oxidant generator coils.

In one or more examples, the graphical user interface 120 can include local conditions 128. The local conditions 128 can include wind speed and direction, temperature, pressure, elevation, humidity, precipitation, sunrise/sunset times, weather forecast, latitude and longitude, etc.). The local conditions 128 can correspond to sensor data obtained via onboard sensors of the client device 118 and/or values obtained from third-party servers and/or web feeds (e.g., really simple syndication (RSS) web feed).

In these or other examples, although not shown in FIG. 1, the client device 118 can include one or more sensors. The one or more sensors can be utilized for directly identifying local conditions (e.g., via physically sampled values of the ambient environment). Additionally or alternatively, the one or more sensors can be utilized for obtaining third-party data corresponding to the local conditions (e.g., using GPS location data of the client device 118 to approximate the local conditions as corresponding to the actual conditions at a nearby third-party measurement sensor/station). In yet another example, the one or more sensors can be utilized to index a data table (e.g., stored within the remote device(s) 102 and/or the memory device of the controller 114) and determine an approximation of the local conditions. For example, the one or more sensors can include a pressure sensor used to sample ambient pressure, which in turn, the scent control apparatus 100 and/or the remote device(s) 102 can use to analyze table values and determine an amount of available oxygen to the scent control apparatus 100. Examples of a sensor can include a global positioning system sensor, pressure sensor, gyroscope, magnetometer, accelerometer, inertial measurement unit, temperature sensor, humidity sensor, infrared sensor, proximity sensor, light sensor, chemical sensor, gas sensor, etc.

Distinct from the client device 118, the remote device(s) 102 can additionally or alternatively include the fob 130. The fob 130 can, like the client device 118, remotely control the scent control apparatus 100. In at least one example, the fob 130 can include a simplified version of the client device 118, a wearable version of the client device 118, a firearm-mounted or bow-mounted version of the client device 118, a surgically implantable version of the client device 118 (e.g., a brain-computer interface), etc. For example, the fob 130 can include one or more buttons or actuators that, in response to user input, cause the fob 130 to transmit a signal—thereby causing the scent control apparatus 100 in turn to perform a certain function. In some implementations, user input to the fob 130 can cause the scent control apparatus 100 to initiate a boost mode in which oxidant output is relatively increased by one or more percentage points (e.g., 10%, 10% to 30%, 20% to 30%, about 50%, between 50% and 100%, or greater than 100%). In one or more examples, the boost mode can help increase scent control (e.g., in a desired moment, such as when an animal is in close proximity to the user). In these or other examples, the fob 130 can include a keychain fob, pendant, necklace, etc. for easy (e.g., no-look and movement-free) control of the scent control apparatus 100.

The network 104 can be any suitable network over which computing devices communicate. Indeed, any of the components shown in FIG. 1 can communicate with each other via the network 104. In these or other examples, the network 104 can include a wireless local area network, wireless area network, wireless personal area network, wide area network, etc. Some particular examples of wireless networks include a Wi-Fi based network, mesh network, BLUETOOTH® network, near-field communication network, low-energy/low power communication network, Zigbee network, Z-wave network, 6LoWPAN network, radio wave-based network, satellite network, LoRa long range communication network, etc. Other forms of the network 104 can include wired connections, such as a USB network, UART network, USART network, I2C network, SPI network, QSPI network, etc.

In one or more examples, the scent control apparatus 100 can include a mounting apparatus 132 to secure or suspend the scent control apparatus 100 in place. As mentioned above, the scent control apparatus 100 can be mountable (e.g., for temporary and/or permanent attachment) to a support element. A support element can include a tree, post, tent pole, bipod (or uni-pod, tripod, etc.) that anchors or otherwise holds in place the scent control apparatus 100. In particular examples, the mounting apparatus 132 can include a fastener element (e.g., a self-tapping screw for manually attaching the mounting apparatus 132 to a tree). In other examples, the mounting apparatus 132 can include a strap, cable lock, etc. for securing the mounting apparatus 132 around a support element. In at least one example, the mounting apparatus 132 includes a clamp (and optionally, one or more shims for fitting smaller-diameter objects like a tent pole). The clamp can compress (and maintain the compression) against a support element to positionally secure the scent control apparatus 100. In specific examples, the mounting apparatus 132 can include a picatinny mount, an Arca-Swiss type mount, etc.

Alternatively, in some examples, the scent control apparatus 100 can be laid on top of or otherwise flush against a support surface (e.g., on top of a ground blind, backpack, foot stool, cooler, ground surface, brush (foliage or undergrowth), etc.). Thus, in a non-suspended position, the bottom surface of the scent control apparatus 100 can be laid adjacent to (e.g., in intimate contact with) a support surface. In these or other examples, the vents 106 are aptly positioned to enable non-suspended configurations in which the scent control apparatus 100 sits atop a support surface. That is, unlike certain prior systems having an intake vent on the bottom surface (and therefore having certain airflow needs at the bottom surface), the scent control apparatus 100 of the present disclosure can include the vents 106 in other locations that better facilitate non-suspended configurations of the scent control apparatus 100 without blocking air intake.

Any of the features, components, and/or parts, including the arrangements and configurations thereof shown in FIG. 1 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 1.

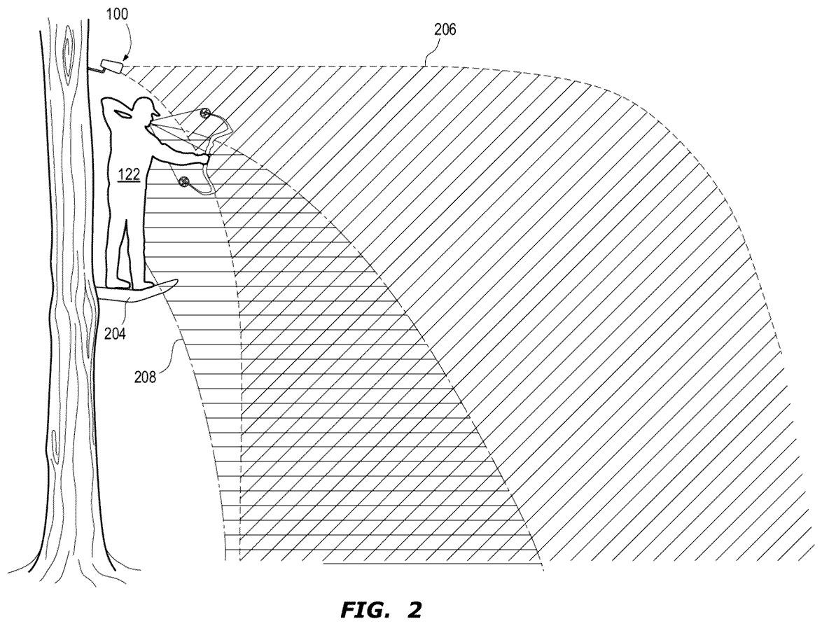

FIG. 2 illustrates an example environment 200 and use case of an example scent control apparatus in accordance with one or more examples of the present disclosure. As shown, the environment 200 includes the scent control apparatus 100 mounted to a tree (e.g., via the mounting apparatus 132). In such a use case, the scent control apparatus 100 can be positioned above a person 202. The person 202, while elevated above ground via a tree stand 204, nonetheless emits a human scent 208. The human scent 208 can stem from gear, clothing, breath exhaled, bodily oils and sweat, etc. Additionally or alternatively, the human scent 208 can generally emit from the person 202 or associated gear, although not necessarily as a by-product of human secretion. For example, the human scent 208 can include scents from detergent, deodorant, body soap or shampoo, shaving cream, cosmetic products, food, oil (e.g., broadhead protectant oil, gun oil), etc. In general, the human scent 208 can be virtually any scent that is foreign to the natural habitat of an animal.

The scent control apparatus 100 as disclosed herein can control the human scent 208 by emitting oxidant 206. The oxidant 206 can be dispersed over the human scent 208 (and beyond) to create a clean, sterile, or treated area in which at least a portion (if not a substantial portion or all) of the human scent 208 is chemically altered, destroyed, or neutralized. Within the treated area, an animal is less apt (if not entirely unable) to detect the human scent 208. For instance, within the treated area, the amount of the human scent 208 not reacted with the oxidant 206 may be of such low concentration that the animal does not perceive the human scent 208 as a threat or does not perceive the person 202 to be in close proximity to the animal. In other instances, the amount of the human scent 208 not reacted with the oxidant 206 may be of such low concentration that the olfactory senses of the animal are incapable of recognizing the presence of the person 202. In other terms, a large portion of the human scent 208 can be so chemically altered, destroyed, or neutralized that the presence of the person 202 is—with respect to the olfactory senses of the animal—undetectable.

In these or other examples, the scent control apparatus 100 can be elevated above the person 202 and oriented such that the oxidant 206 falls downwind and toward the ground, thereby falling on top of and reacting with the human scent 208. However, other configurations and implementations are herein contemplated—particularly for different use cases and different environments. For example, in a spot-and-stalk scenario in which a hunter is often crawling on the ground or laying in a prone position, the scent control apparatus 100 can be set on top of (or leaned against) a backpack adjacent to the hunter such that the scent control apparatus 100 is oriented horizontal and/or upward to treat—in a different fashion than shown in FIG. 2—a different area around the hunter already positioned at ground level. Other use cases can, for instance, include ground blinds, tripods, and various other mounted or non-mounted configurations. Thus, a variety of different implementations and use-case scenarios for the scent control apparatus 100 are herein contemplated.

Any of the features, components, and/or parts, including the arrangements and configurations thereof shown in FIG. 2 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 2.

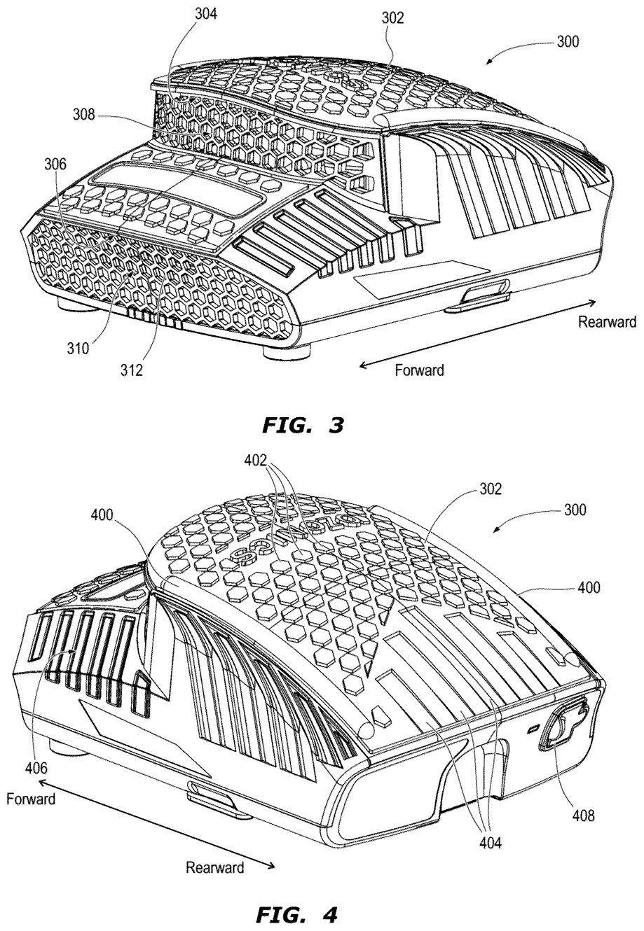

FIGS. 3-9 respectively illustrate front perspective, rear perspective, side, front, rear, bottom, and top views of an example scent control apparatus 300 in accordance with one or more examples of the present disclosure. In these or other examples, the scent control apparatus 300 can be the same as or similar to the scent control apparatus 100 discussed above.

As shown, the scent control apparatus 300 can include a housing 302. The housing 302 can include an enclosure, body, or shell that houses various components of the scent control apparatus 300. The housing 302 can include a variety of materials, coatings, overmoldings, etc. For example, the housing 302 can include a plastic, metal, rubber, and/or composite material. In particular examples, the housing 302 can include a weather resistant housing or protective cover at least partially enclosing the components of the scent control apparatus 300. In some examples, the housing 302 can include various water-shedding features designed to direct water in a certain way (which features are discussed more below).

In at least one example, the housing 302 defines housing openings 304, 306. The housing openings 304, 306 can include apertures, slits, through-holes, passageways, etc. that fluidly couple an internal volume of the scent control apparatus 300 defined by the housing 302 and the ambient environment external to the scent control apparatus 300. In particular examples, the housing openings 304, 306 correspond to an intake vent 308 and a flow-restricted output vent 310, respectively. Ambient air is brought into the scent control apparatus 300 through the intake vent 308. Oxidant is emitted out into the ambient environment through the flow-restricted output vent 310. The intake vent 308 and the flow-restricted output vent 310 can, therefore, be the same as or similar to the vents 106 discussed above.

In one or more examples, the intake vent 308 is an upper vent and the flow-restricted output vent 310 is a lower vent relative to the intake vent 308 (albeit alternative arrangements are herein contemplated). That is, in some examples, the intake vent 308 is elevated (e.g., higher or above) relative to the flow-restricted output vent 310. In such examples, the intake vent 308 can be positioned farther away from a user (e.g., so as to reduce perceived noise by a user positioned underneath the scent control apparatus 300 when in a tree stand, for instance). In some examples, both the intake vent 308 and the flow-restricted output vent 310 are forward-facing (e.g., for convenience and ease of positioning/orienting the scent control apparatus 300 relative to the downwind direction). In other examples, at least one of the intake vent 308 or the flow-restricted output vent 310 faces rearward or sideways (oblique or non-parallel to the forward and rearward directions).

As mentioned, oxidant can be emitted through the flow-restricted output vent 310. The term “flow-restricted” refers to the constricted nature of the flow-restricted output vent 310 relative to the intake vent 308. For example, the flow-restricted output vent 310 can be partially covered, blocked, restricted, or limited. Additionally or alternatively, the size of the flow-restricted output vent 310 can be smaller than the intake vent 308 (e.g., tapered, reduced, narrowed, etc.). However, the term “flow restricted” is not limited to a description of the output vent. For instance, a fluid flow path (e.g., an intake path, an air path, an airflow route, etc.) can be constricted anywhere inside the housing 302 and/or by one or more other components in addition to, or alternatively to, a constriction at the flow-restricted output vent 310. As will be discussed further below, there are a number of possible advantages for implementing the flow-restricted output vent 310 in this way, particularly with respect to inducing certain flow characteristics.

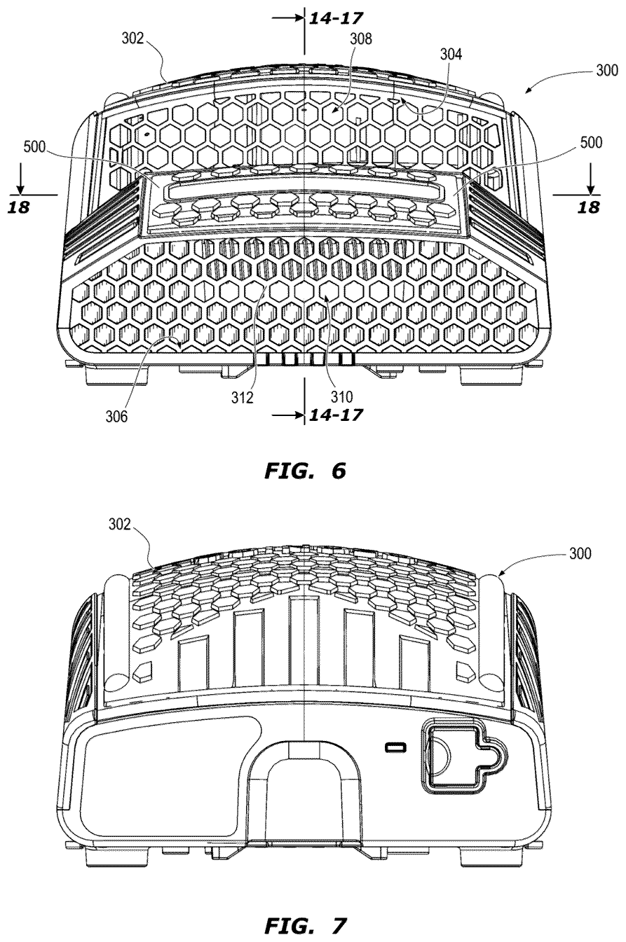

In one example, a permeable cover 312 can be disposed over the flow-restricted output vent 310. The permeable cover 312 can include at least one of a pass-through grate, grille, barrier, screen, etc. The permeable cover 312 can, thus, at least partially cause flow-restriction at the flow-restricted output vent 310. For example, and as particularly shown in FIG. 6, the permeable cover 312 can include a webbing (e.g., arranged in a honeycomb structure) that defines a plurality of interstitial apertures. The webbing (or support structure defining the interstitial apertures) can at least partially inhibit fluid flow out of the flow-restricted output vent 310 because the webbing is positioned directly between the flow-restricted output vent 310 and the ambient environment, like an obstacle that fluid flow must traverse around in order to exit the flow-restricted output vent 310. In some examples, a portion of the permeable cover 312 is non-permeable, solid, or otherwise designed to exclude the passage of fluid (see, e.g., the portion of the permeable cover 312 surrounding a perimeter of the flow-restricted output vent 310). In at least one implementation, the solid portion of the permeable cover 312 can also serve to constrict, limit, or size-reduce the flow-restricted output vent 310 relative to the second aperture 306. In some examples, the permeable cover 312 can induce turbulent flow conditions (e.g., from backpressure and/or stream separation in and around the structure of the permeable cover 312). In one or more examples, the permeable cover 312 can also be integrally formed with an interior chute (while in other examples, separate from the interior chute).

As mentioned above, the housing 302 (and/or associated overmolding) can include a variety of exterior surface features, including moisture-shedding features, grip features, aesthetic features, etc. As some examples, the housing 302 can include protrusions 400. The protrusions 400 can include elongated portions of the housing 302 arranged as bumps, projections, lips, bulges, etc. that extend outward (i.e., jut away from) the surface of the housing 302. In one or more examples, the protrusions 400 can be positioned to guide and shed moisture from the housing 302 (e.g., as a water relief route or embankment). For instance, the protrusions 400 can be positioned on one or more top surfaces of the housing 302 along the side edges. In this way, the protrusions 400 can help redirect moisture away from at least one of the intake vent 308 or the flow-restricted output vent 310.

The housing 302 can include stippling 402 on the top surface of the scent control apparatus 300. The stippling 402 (e.g., localized bumps or texturing) of the housing 302 along with the curved profile of the housing 302 can help alleviate the gathering of moisture on the top surface of the scent control apparatus 300. In some examples, the stippling 402 can help distribute and control moisture flow as it moves across the housing 302. In particular examples, the stippling 402 can provide increased ease of grip for maneuvering or handling the scent control apparatus 300 (as can ribs 406 positioned, for instance, along the side of the housing 302).

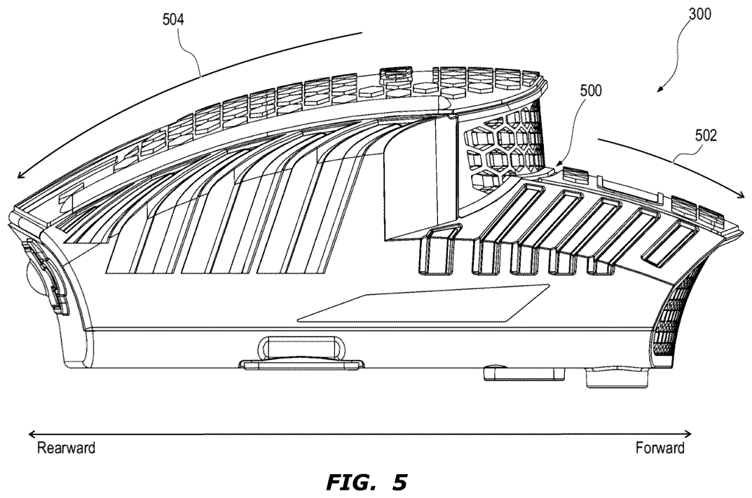

The housing 302 can include raceways 404 (see FIG. 4) and channels 500 (see FIGS. 5-6). The raceways 404 and the channels 500 can provide designated moisture pathways for shedding off of the top surfaces of the scent control apparatus 300. The raceways 404 and the channels 500 can, based on their positioning, catch various portions of moisture. In some examples, the raceways 404 and the channels 500 can then route the moisture off the scent control apparatus 300 in predetermined directions (e.g., water shedding directions 502, 504). The water shedding directions 502, 504 can, in some examples, vary depending on the orientation of the scent control apparatus 300 relative to the force of gravity acting upon the scent control apparatus 300. For instance, if the scent control apparatus 300 is tilted sufficiently forward, the water shedding direction 504 can change to a forward direction (similar to the water shedding direction 502). As another example, side-to-side moisture shedding can also be achieved. For instance, the top surface of the housing 302 can include a crown or rounded top such that moisture sheds from the top portion to one of the side portions of the housing 302. In these or other examples, moisture shedding—particularly moisture shedding away from the flow-restricted output vent 310—can reduce interaction between the oxidant and moisture.

In one or more examples, the housing 302 can define a power port 408. The charge port 408 can receive a power input to charge a power supply inside the scent control apparatus 300 and/or provide a direct power source to the scent control apparatus 300. In one or more examples, the charge port 408 can include a socket, adapter port, USB-port, USB-C port, A/C plug, or other suitable connector for receiving power input to the scent control apparatus 300. In at least one example, the charge port 408 can be weather resistant when sealed, closed, or shut.

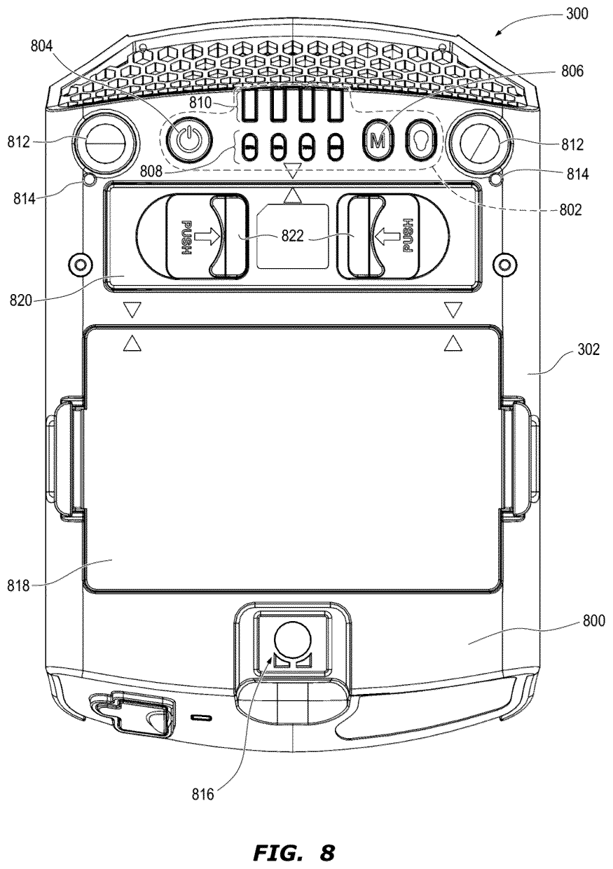

FIG. 8 depicts some example features located on a bottom surface 800 of the scent control apparatus 300. For example, the scent control apparatus 300 can include a control interface 802. The control interface 802 can be the same as or similar to the control interface 116 discussed above. In particular examples, the control interface 802 can include a power button 804 (e.g., to turn the scent control apparatus 300 on and off). When activated or pushed, the power button 804 can cause the power supply for the scent control apparatus 300 to provide power to the fan and the detachable ozone generator coil. In contrast, when deactivated (e.g., released, unpressed or switched off), the power button 804 can cause the power supply for the scent control apparatus 300 to cut power to the fan and the detachable oxidant generator coil.

The control interface 802 can include a mode button 806. The mode button 806 can include any actuator (e.g., dial, switch, button, rocker, toggle, etc.) that, when actuated, can cause the scent control apparatus 300 to switch between various operating modes (e.g., “Boost,” “Hyperboost,” “Locker,” “Driwash,” “Standard”). Additionally or alternatively, the mode button 806 can include one or more actuators that, when actuated, can cause the scent control apparatus 300 to initiate a standby mode (in which there is power provided to at least one of the fan or the detachable oxidant generator coil, but the fan is not rotating). In another example, the mode button 806 can include one or more actuators that, when actuated, can cause the scent control apparatus 300 to transition from the standby mode to an operating mode (and/or vice-versa). In some examples, the various operating modes are indicated via mode indicators 810 (which respectively illuminate according to the selected operating mode). In particular examples, the mode indicators 810 are color coded according to operating mode (e.g., green for a first mode, blue for a second mode, and so forth).

The control interface 802 can include battery status indicators 808. The battery status indicators 808 can respectively illuminate according to a charge level of the power supply (e.g., at approximations of 100% charge level, 75% charge level, 50% charge level, and 25% charge level). In particular examples, the battery status indicators 808 can also be color coded according to the charge level (e.g., green for 100% charge level, yellow for 75% charge level, orange for 50% charge level, and red for 25% charge level).

In one or more examples, the scent control apparatus 300 can include light elements 812. The light elements 812 can be positioned on the bottom surface of the scent control apparatus 300 (e.g., for providing above-head illumination when mounting the scent control apparatus 300 and/or when needing convenient light for visibility in low-light or nighttime settings). Such bottom-facing lighting form the light elements 812 can be particularly advantageous in the early morning and late evening hours when a user may need hands-free lighting to perform tasks in a tree stand (e.g., maneuver/retrieve gear, nock an arrow, adjust safety harness, etc.). In some examples, the light elements 812 can be low light or big-game friendly (e.g., red light, green light, etc.) The light elements 812, in particular examples, can include various illumination modes. In these or other examples, an illumination button of the control interface 802 can control the light elements 812.

In some examples, the scent control apparatus 300 can include weep holes 814. The weep holes 814 can be positioned in a variety of locations (e.g., adjacent to the light elements 812, as shown). In these or other examples, the weep holes 814 can help evacuate moisture from inside the scent control apparatus 300. Various internal components can also help facilitate interior moisture evacuation and/or direct moisture to the weep holes 814 for internal moisture evacuation. In certain implementations, internal moisture evacuation can help increase the efficiency and longevity of the detachable oxidant generator coil (e.g., by reducing or preventing moisture buildup, and particularly by inhibiting moisture contact with the oxidant generator coil and oxidant(s) generated therefrom).

Further shown in FIG. 8, the scent control apparatus 300 can include a mounting receptacle 816. The mounting receptacle 816 can receive the mounting apparatus 132 discussed above in relation to FIG. 1. The mounting receptacle 816 can include various connectors, attachment elements, threaded engagements, locking engagements, etc. for securely attaching the scent control apparatus 300 to the mounting apparatus 132.

The scent control apparatus 300 can also include a battery cover 818 disposed over the power supply compartment (and associated power supply) of the scent control apparatus 300. In some examples, the battery cover 818 can include alignment indicators to ensure proper alignment and attachment of the battery cover 818 to the scent control apparatus 300. The battery cover 818 can also include tabs for user manipulation to unlock, release, or detach the battery cover 818 from the scent control apparatus 300.

In one or more examples, the scent control apparatus 300 can include an oxidant generator coil cartridge 820. The oxidant generator coil cartridge 820 can include the detachable oxidant generator coil 112 discussed above in relation to FIG. 1. For example, the oxidant generator coil cartridge 820 can include a housing, shell, holder, retaining mechanism, etc. for the detachable oxidant generator coil 112. Thus, at the time of replacement or substitution, the detachable oxidant generator coil 112 can be switched out along with the oxidant generator coil cartridge 820 (e.g., insert a whole different oxidant generator coil cartridge and a different oxidant generator coil). Alternatively, the detachable oxidant generator coil 112 can be removed from the oxidant generator coil cartridge 820 itself such that a new oxidant generator coil is attached to the old cartridge, and both are re-inserted into the scent control apparatus 300. Regardless of the replacement method, the oxidant generator coil cartridge 820 and associated oxidant generator coil can advantageously impart improved convenience and ease of self-maintenance over prior systems and methods in the art that implement ozone generating element(s) not designed for removal. Moreover, and as will be discussed below, the oxidant generator coil cartridge 820 can position the detachable oxidant generator coil 112 in a direct flow path of air such that the detachable oxidant generator coil 112 is arranged in alignment (e.g., in a straight or linear relationship) between a fan and an exit chute adjacent the flow-restricted output vent 310.

A variety of implementations for detaching and/or re-attaching the oxidant generator coil cartridge 820 are herein contemplated. In some examples, the oxidant generator coil cartridge 820 utilizes a release mechanism 822. The release mechanism 822 can be manipulated for unlocking, releasing, or detaching the oxidant generator coil cartridge 820 from the scent control apparatus 300. For example, the release mechanism 822 can include release tabs that, when pinched or pushed together, cause the oxidant generator coil cartridge 820 to release from the scent control apparatus 300. The release mechanism 822 can, in some examples, include a recess, raised walls, etc. for enabling convenient, ergonomic actuation with gloved hands (e.g., having thick insulating gloves). In certain implementations, the release mechanism 822 can be manipulated for re-attaching and securing the oxidant generator coil cartridge 820 within the scent control apparatus 300. In other implementations, the release mechanism 822 are self-movable as the oxidant generator coil cartridge 820 is inserted into and slid into a fully-seated position. That is, in some examples, the release mechanism 822 need not be manually actuated by the user in order to lock the oxidant generator coil cartridge 820 into position within the scent control apparatus 300. In these or other examples, the oxidant generator coil cartridge 820 can include alignment indicators to ensure proper alignment and attachment of the oxidant generator coil cartridge 820 to the scent control apparatus 300.

Any of the features, components, and/or parts, including the arrangements and configurations thereof shown in FIGS. 3-9 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 3-9.

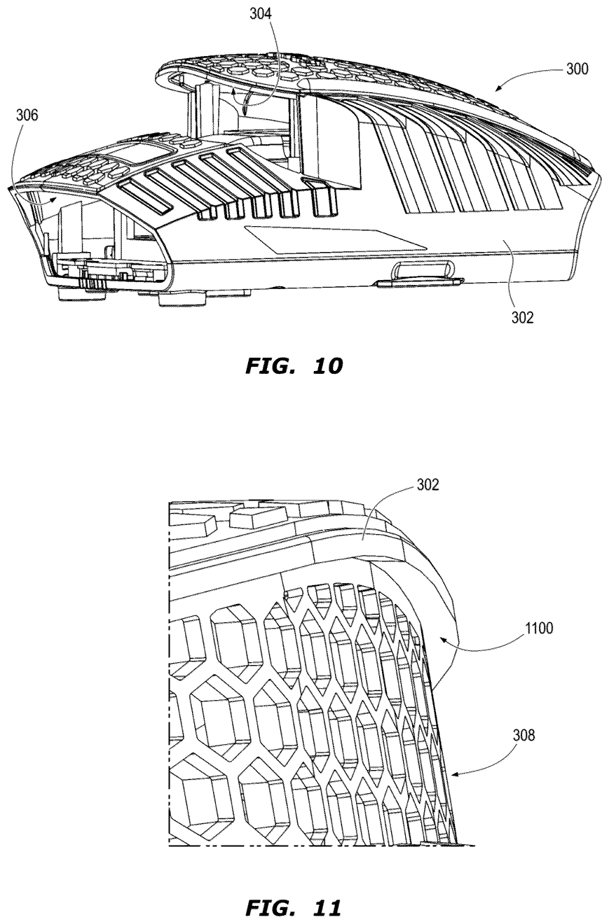

FIG. 10 illustrates examples of the first aperture 304 and the second aperture 306 defined by the housing 302 of the scent control apparatus 300 in accordance with one or more examples of the present disclosure. From FIG. 10, and in comparison to FIG. 6 (showing the permeable cover 312), it is visually apparent that the permeable cover 312 can reduce an exit opening for oxidant to pass through the second aperture 306—hence the output vent (shown in FIG. 6) is the flow-restricted output vent 310. As will be discussed more below, the permeable cover 312 (omitted here for illustration purposes of the second aperture 306), can induce backpressure inside the scent control apparatus 300 and/or induce turbulent flow conditions. At least one of back pressure or turbulent flow conditions can help increase air contact with the detachable oxidant generator coil 112 inside the scent control apparatus 300 and improve oxidant generation efficiency.

Any of the features, components, and/or parts, including the arrangements and configurations thereof shown in FIG. 10 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 10.

FIG. 11 illustrates a close-up view of an example exterior water-shedding feature in accordance with one or more examples of the present disclosure. As shown in FIG. 11, the housing 302 defines an overhang 1100 extending over an upper perimeter portion of the intake vent 308. The overhang 1100 can, in some examples, help reduce or prevent moisture from flowing into the intake vent 308. For instance, moisture can drip or run off the overhang 1100 in a manner that bypasses the intake vent 308 due to the length of the top housing surface extending forward beyond the intake vent 308. In other words, the intake vent 308 can be recessed inwards (or rearward) relative to the outer edge of the overhang 1100, thereby creating a profiled eyelid protecting against water infiltration into the intake vent 308. In some examples, the overhang 1100 is at least about 1 mm to about 100 mm, about 2 mm to about 50 mm, about 5 mm to about 20 mm, or about 10 mm to about 75 mm.

Any of the features, components, and/or parts, including the arrangements and configurations thereof shown in FIG. 11 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 11.

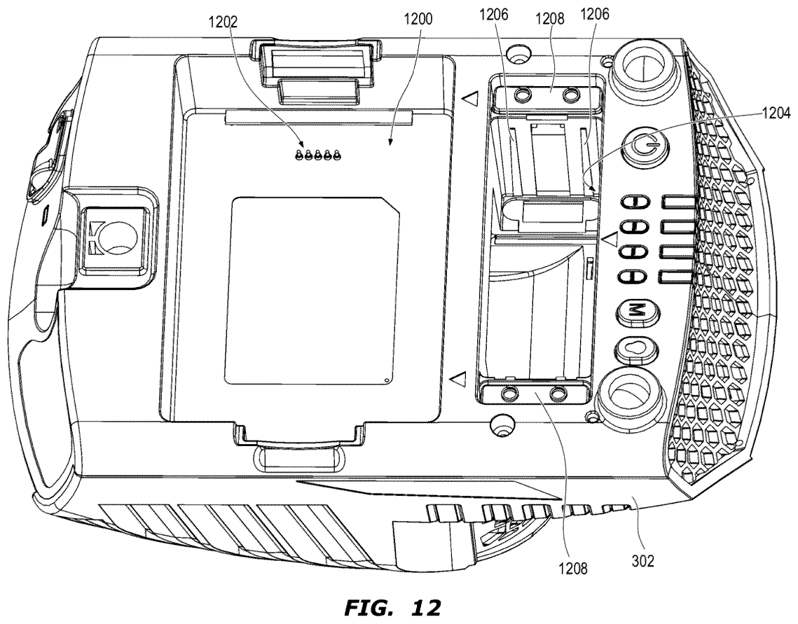

FIG. 12 illustrates a bottom perspective view of the scent control apparatus 300 having the battery cover 818 (and associated power supply) and the oxidant generator coil cartridge 820 removed for illustration purposes in accordance with one or more examples of the present disclosure.

As shown, the scent control apparatus 300 can include a battery compartment 1200 defined by the housing 302. In some examples, the battery compartment 1200 can be sized, shaped, and/or positioned to create a more stable or balanced apparatus. For example, the battery compartment 1200 can be centered across the width of the scent control apparatus 300 to help provide increased balance. As another example, the battery compartment 1200 can have a shallower depth that, in combination with the width-spanning dimensions, is sized and shaped to receive a particular size configuration of a power supply. For example, in specific implementations, the battery compartment 1200 is sized and shaped for a single row of batteries (e.g., a 4×1 battery cell (e.g., pack) configuration, a 6×1 battery cell configuration, an 8×1 battery cell configuration, etc.). The single row configuration of battery cells can, in some examples, lower the overall temperature of the power supply by increasing the surface area (thereby more efficiently cooling the battery cells during charge/discharge cycles). Other battery cell configurations, including vertically stacked rows of battery cell configurations, are herein contemplated.

Further shown, the battery compartment 1200 can include a connector 1202. The connector 1202 can include electrical connections of the scent control apparatus 300 for electrically coupling with a power supply. In some examples, the connector 1202 includes pogo pins. The pogo pins can have a variety of structural and material configurations. In particular examples, the pogo pins are gold plated to help reduce or prevent oxidation. As another example, the pogo pins can include a sufficient length to enable travel (or increased travel) that helps maintain electrical connectivity in a variety of use cases, including on-the-go implementations in which a user is operating the scent control apparatus 300 while moving (and therefore causing the scent control apparatus 300 to jostle, bounce, vibrate, etc.). Lengthened pogo pins can, in some examples, reduce or eliminate movement of the power supply within the battery compartment 1200. In at least one example, the battery compartment 1200 can be keyed to ensure the power supply is properly mounted to connect with the connector 1202.

In some examples, the housing 302 can define an oxidant generator coil cartridge compartment 1204 for the oxidant generator coil cartridge 820. The oxidant generator coil cartridge compartment 1204 can thus be sized and shaped to receive the oxidant generator coil cartridge 820. For example, the oxidant generator coil cartridge compartment 1204 can be sized and shaped such that the oxidant generator coil cartridge 820, when inserted into the scent control apparatus 300, positions the detachable oxidant generator coil 112 in a direct flow path between the fan and the exit chute leading to (or otherwise fluidly connected to) the flow-restricted output vent 310. In at least one example, the oxidant generator coil cartridge compartment 1204 can include housing keys 1206 defined by the housing 302. The housing keys 1206 can include connection features (e.g., slots, protrusions, guides, etc.) that can dictate directionality or orientation of insertion for the oxidant generator coil cartridge 820 into the oxidant generator coil cartridge compartment 1204. In some examples, the enforced directionality or orientation of the oxidant generator coil cartridge 820 can ensure proper electrical connection of the oxidant generator coil cartridge 820 to the scent control apparatus 300 (e.g., for energizing the detachable oxidant generator coil 112).

In at least one example, one or more portions of the oxidant generator coil cartridge compartment 1204 can include a biasing mechanism and/or biased contact surfaces (e.g., to help seat the oxidant generator coil cartridge 820 in place, maintain electrical connectivity with the oxidant generator coil cartridge 820, and/or reduce play of the oxidant generator coil cartridge 820 within the oxidant generator coil cartridge compartment 1204). For example, biased contact surfaces 1208 can be biased (e.g., spring-loaded) to contact the oxidant generator coil cartridge 820. In one or more examples, the biased contact surfaces 1208 can actuate an electrical switch (not shown) when the oxidant generator coil cartridge 820 is inserted into the oxidant generator coil cartridge compartment 1204. For instance, a plunger (not shown) integrally formed with the biased contact surfaces 1208 can actuate an electrical switch that enables power to flow to the oxidant generator coil cartridge 820 when the oxidant generator coil cartridge 820 is inserted into the oxidant generator coil cartridge compartment 1204.

Any of the features, components, and/or parts, including the arrangements and configurations thereof shown in FIG. 12 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 12.

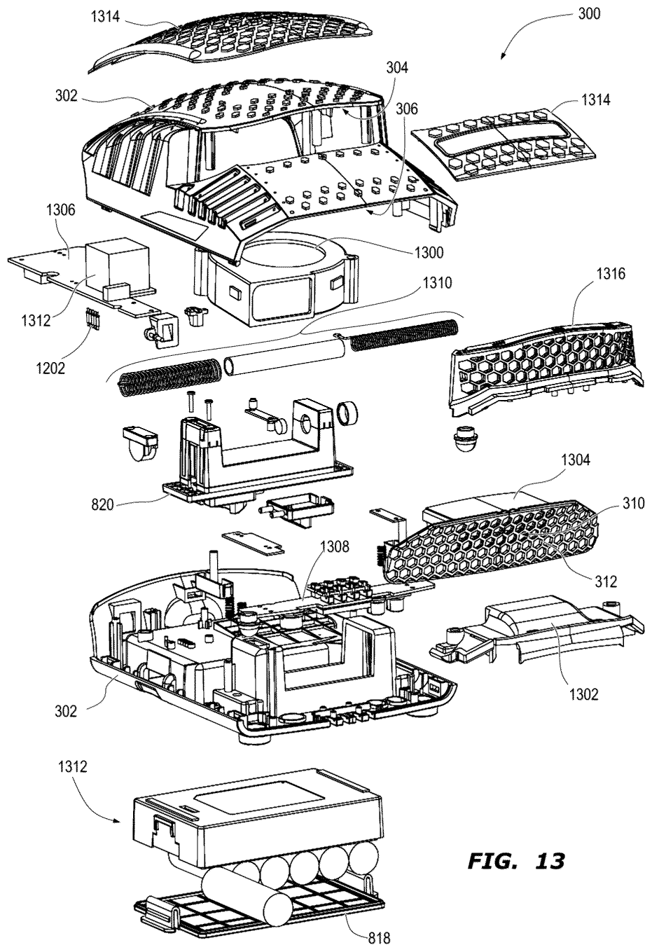

FIG. 13 illustrate an exploded view of the scent control apparatus 300 in accordance with one or more examples of the present disclosure. FIG. 13, therefore, depicts many of the components previously discussed above. More of the internal components of the scent control apparatus 300 are now introduced or formally referenced.

For example, the scent control apparatus 300 can include a fan 1300, a manifold 1302, and a chute 1304. The fan 1300 can be the same as or similar to the fan 110 discussed above in relation to FIG. 1. For instance, the fan 1300 can include a cage fan (e.g., that generates less noise than other fan types). In particular examples, the fan 1300, the manifold 1302, and the chute 1304—as well as the placement of these components relative to an oxidant generator coil 1310—can work together to improve operational efficiency (e.g., decrease power consumption and/or increase oxidant generation per expended energy), reduce sound energy, and/or increase oxidant production. In at least one example, these components can also work together to reduce internal oxidant buildup (thereby decreasing internal oxidation and decreasing internal buildup of nitric acid residue from hydroxyl exposure).

In some examples, the manifold 1302 includes a hood, tube, duct, conduit, etc. for directing (or guiding) airflow from the fan 1300 toward the fan 1300. In at least one example, the manifold 1302 is partially open and defines a partial flow path (e.g., an upper boundary and/or side boundary of an airflow path).

In some examples, the chute 1304 can include another hood, tube, duct, conduit, etc., except for directing oxidant from the oxidant generator coil 1310 toward the flow-restricted output vent 310. In at least one example, the chute 1304 is a discrete component from the manifold 1302, as these components can control different areas of a flow path.

In these or other examples, the oxidant generator coil 1310 can be the same as or similar to the detachable oxidant generator coil 112 discussed above in relation to FIG. 1. In some examples, the oxidant generator coil 1310 can include an inner and outer coil (e.g., precision wound metal coils). In at least one embodiment, the inner and outer coils are formed of stainless steel (e.g., 316 passivated stainless steel). Additionally, the oxidant generator coil 1310 can include an insulating tube positioned between the inner and outer coils. The insulating tube can include one or more of a variety of materials (e.g., a quartz crystal material). The positional relationship of the fan 1300, the manifold 1302, and the chute 1304 relative to the oxidant generator coil 1310 is discussed more below.

Further shown in FIG. 13, the scent control apparatus 300 can include a main printed circuit board (PCB) 1306. The main PCB 1306 can include a controller for controlling operation of various components of the scent control apparatus 300 (e.g., the fan 1300 and the oxidant generator coil 1310). In some examples, the controller of the main PCB 1306 can be the same as or similar to the controller 114. For example, the main PCB 1306 can include a processor and/or a memory device (e.g., for storing computer-executable instructions that, when executed by the processor, cause the processor to perform certain functions).

In one or more examples, the scent control apparatus 300 can include a PCB 1308. The PCB 1308 can include a controller for controlling operation of various components of the scent control apparatus 300 (e.g., the fan 1300, the oxidant generator coil 1310, the light elements 812, etc.) via user input to the control interface 802 discussed above in relation to FIG. 8. The controller of the PCB 1308 can be the same as or similar to the controller 114 discussed above in relation to FIG. 1. For example, the PCB 1308 can include a processor and/or a memory device (e.g., for storing computer-executable instructions that, when executed by the processor, cause the processor to perform certain functions).

In one or more examples, various components of the scent control apparatus 300 can include a protective coating. For example, the main PCB 1306 and the PCB 1308 can include a protective coating. The protective coating can include various epoxies, sealants, etc. In these or other examples, the protective coating can protect sensitive components from moisture, oxidation, and/or hydroxyl radicals forming nitric acid granules thereon.

In addition, FIG. 13 shows the scent control apparatus 300 can include a power supply 1312. The power supply 1312 can be the same as or similar to the power supply 108 discussed above in relation to FIG. 1. In at least one example, the power supply 1312 can include a rechargeable battery pack that includes one or more battery cells. In one or more examples, the power supply 1312 can include a heating film positioned around one or more battery cells (e.g., to combat efficiency losses in colder weather). For instance, the power supply 1312 can include a polyamide heating film wrapped around the battery cells inside the power supply 1312.

In some examples, the scent control apparatus 300 can include overmold portions 1314. The overmold portions 1314 can provide texturing, grip, aesthetics, and/or moisture shedding features.

The scent control apparatus 300 can, additionally or alternatively, include a cover 1316. The cover 1316 can be disposed over the first aperture 304. The cover 1316 can inhibit debris or objects from gaining access into the scent control apparatus 300 through the first aperture 304. The cover 1316, in some examples, differs from the permeable cover 312, however. For instance, the cover 1316 may not constrict or reduce the first aperture 304 to the extent that the permeable cover 312 can reduce the second aperture 306. Thus, in some embodiments, air intake is not inhibited (or least not substantially throttled) by the cover 1316. Additionally, in at least one example, the cover 1316 can be removably detached from (and reattached to) the housing 302 over the first aperture 304 (whereas, in some examples, the permeable cover 312 can be formed integral to or otherwise affixed to the chute 1304).

Any of the features, components, and/or parts, including the arrangements and configurations thereof shown in FIG. 13 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 13.

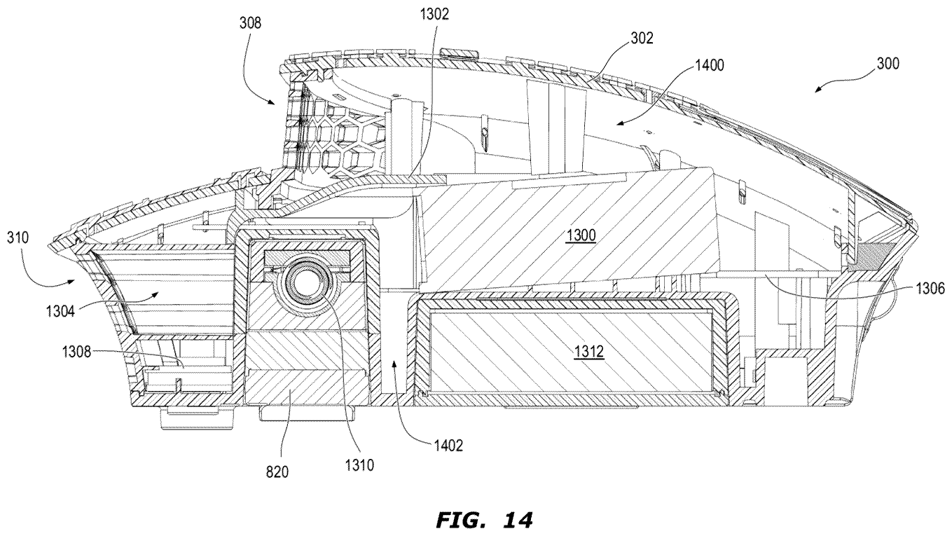

FIGS. 14-18 illustrate various cross-sectional views of the scent control apparatus 300 in accordance with one or more examples of the present disclosure. In these views, an interior volume 1400 is exposed for viewing the various internal components of the scent control apparatus 300. The interior volume 1400 is, in some examples, defined by the housing 302.

As shown, the scent control apparatus 300 can include the fan 1300 configured to intake air through the intake vent 308 (e.g., the upper vent) and force the air along a direct flow path toward the flow-restricted output vent 310 (e.g., the lower vent). In one or more examples, the fan 1300 can be secured to the housing 302 at a predetermined pitch or otherwise oriented to flow air directly (i.e., in a substantially straight or linear path) toward the oxidant generator coil 1310 and thereafter into the chute 1304 leading to the flow-restricted output vent 310. In these or other examples, the fan 1300 can be positioned above the power supply 1312 and adjacent to the main PCB 1306 (albeit other internal configurations are herein contemplated). At least a top portion (or edge) of the fan 1300 can be secured to the manifold 1302 for guiding airflow onto the oxidant generator coil 1310.

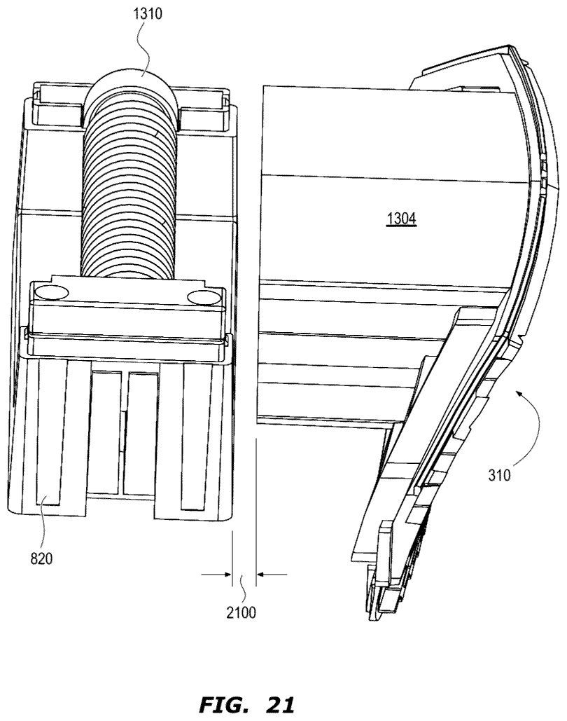

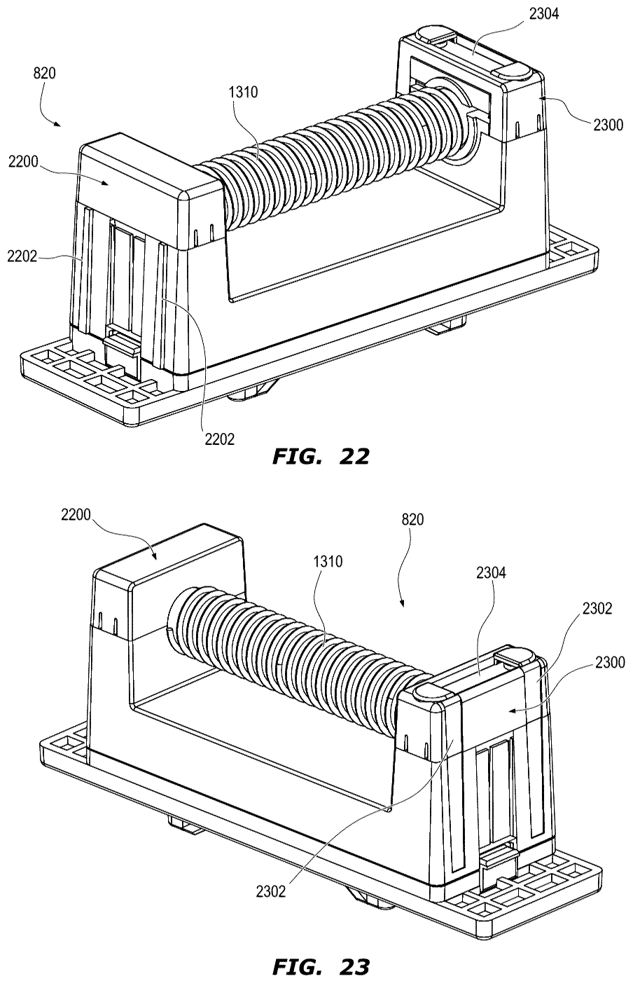

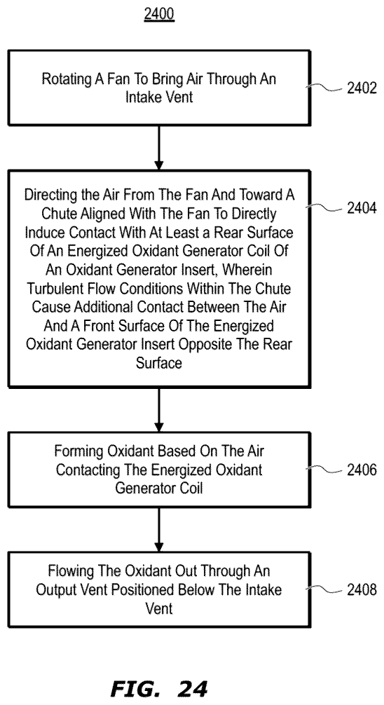

Additionally shown, the oxidant generator coil 1310 can be secured to the housing 302 via the oxidant generator coil cartridge 820 and positioned in the direct flow path (which flow path is visually referenced in FIG. 20). For example, the oxidant generator coil cartridge 820 can be removably secured to the housing 302 to position the oxidant generator coil 1310 between the fan 1300 and the chute 1304. In these or other examples, when the oxidant generator coil cartridge 820 is inserted into position in the scent control apparatus 300, the oxidant generator coil 1310 can be suspended vertically at a height between the manifold 1302 and the oxidant generator coil cartridge 820. Further, in some examples, when the oxidant generator coil cartridge 820 is inserted into position in the scent control apparatus 300, the oxidant generator coil 1310 can be laterally suspended via end portions of the oxidant generator coil cartridge 820 (see FIGS. 22-23) and thereby span a horizontal distance across a majority of the fan exit opening and the entrance of the chute 1304.

In one or more examples, the scent control apparatus 300 can include an air gap 1402 positioned between the power supply 1312 and the oxidant generator coil cartridge 820. The air gap 1402 can be defined by the interior surfaces of the housing 302. In these or other examples, the air gap 1402 can help facilitate a cooling effect for the power supply 1312. In particular examples, the air gap 1402 can help the power supply 1312 to more rapidly cool (e.g., dissipate heat) during charge/discharge cycles. In one or more examples, the air gap 1402 can also distance the power supply 1312 from the oxidant generator coil 1310, which can prolong the life of the oxidant generator coil 1310 and/or reduce heat transfer between the oxidant generator coil 1310 and the power supply 1312.

Any of the features, components, and/or parts, including the arrangements and configurations thereof shown in FIGS. 14-18 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 14-18.

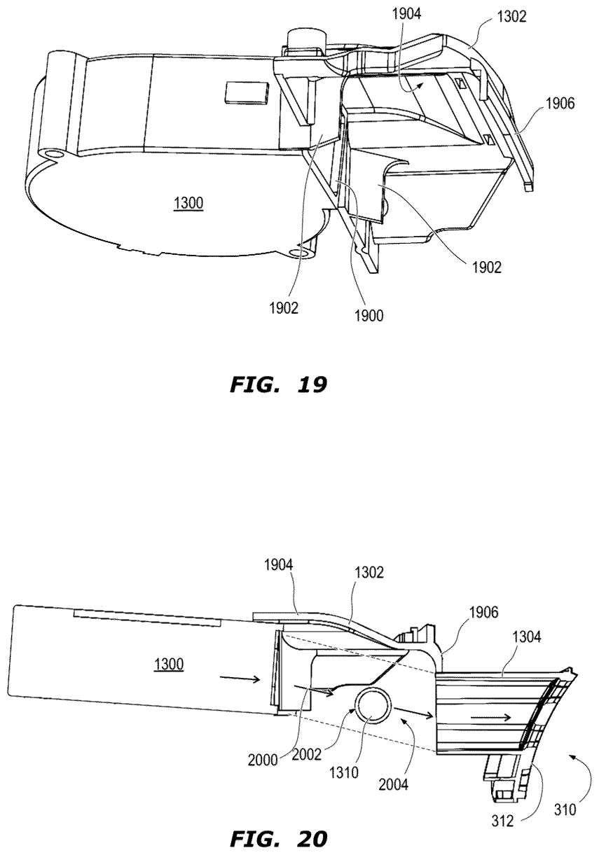

FIG. 19 illustrates an example of the fan 1300 and the manifold 1302 of the scent control apparatus 300 in accordance with one or more examples of the present disclosure. As shown, the fan 1300 can include a fan exit opening 1900 for forced air to exit the fan 1300. Upon exiting the fan 1300, the manifold 1302 can guide air toward the oxidant generator coil 1310 (as more particularly shown in FIG. 20). For example, the manifold 1302 can include walls 1902 positioned at opposing sides of the fan exit opening 1900. The walls 1902, in some examples, can flare outward allowing expansion of air from the fan exit opening 1900 and/or for inducing certain flow conditions (e.g., laminar flow conditions of air between the fan exit opening 1900 and the oxidant generator coil 1310). In some examples, the walls 1902 can direct airflow along a certain flow path toward the oxidant generator coil 1310.

An upper portion 1904 of the manifold 1302 can likewise guide or direct airflow along a certain flow path toward the oxidant generator coil 1310. In some examples, where the fan 1300 is elevated relative to the chute 1304, the upper portion 1904 can help direct airflow downward at an angle. Additionally or alternatively, the upper portion 1904 can induce certain flow conditions before and/or after the oxidant generator coil 1310. For example, a part of the upper portion 1904 positioned rearward of the oxidant generator coil 1310 can induce laminar flow conditions, but part of the upper portion 1904 (e.g., the lip 1906) positioned forward of the oxidant generator coil 1310 can induce turbulent flow conditions. For instance, the lip 1906 can help direct a portion of the air above the oxidant generator coil 1310 to bounce back or curl rearward to contact the oxidant generator coil 1310. In some examples, the lip 1906 can help direct oxidant (generated upon air contact with the oxidant generator coil 1310) to pass into the chute 1304.

Any of the features, components, and/or parts, including the arrangements and configurations thereof shown in FIG. 19 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 19.

FIG. 20 illustrates a side cross-sectional view of an example positional relationship between the fan 1300, the manifold 1302, and the chute 1304 of the scent control apparatus 300 in accordance with one or more examples of the present disclosure. In particular, FIG. 20 expressly depicts a flow path 2000 of airflow proceeding directly in an approximately straight or linear path from the fan exit opening 1900 toward the oxidant generator coil 1310. In these or other examples, the fan 1300 and the oxidant generator coil 1310 can be aligned relative to each other to enable the flow path 2000. For example, the flow path 2000 can proceed from the fan exit opening 1900 to a rear surface 2002 of the oxidant generator coil 1310 closest to the fan 1300—without any bends, turns, elbows, or twists in the flow path 2000. In some examples, this portion of the flow path 2000 can be substantially laminar flow (e.g., to lower energy demand for the fan 1300).

Various design configurations of the fan 1300, the manifold 1302, the flow-restricted output vent 310, and/or the permeable cover 312 relative to the oxidant generator coil 1310 can impart certain advantages. For example, in some implementations, the foregoing components can induce air contact with the front surface 2004 (positioned closest to the flow-restricted output vent 310 and opposite the rear surface 2002). By facilitating air contact with the front surface 2004, oxidant generation can be improved. For instance, surface utilization of the oxidant generator coil 1310 can be increased from about 40%-60% to approximately 100% surface utilization (i.e., 360-degree air contact all the way around the oxidant generator coil 1310). That is, instead of air contact being limited to the rear surface 2002, the various components of FIG. 20 can work together to enable air contact with the front surface 2004 as well.

In at least some examples, the various components of FIG. 20 can work together to reduce internal oxidant levels. For example, instead of oxidant building up or pooling inside the scent control apparatus 300, oxidant can be effectively (and efficiently) discharged—with little or no internal oxidant buildup inside the interior volume 1400. Experimental data, for instance, suggests that the various components of FIG. 20 can work together to reduce internal oxidant levels down to about 0 to 10 parts per million (PPM), about 1 to 8 PPM, or about 2 to 5 PPM (whereas prior systems may include internal oxidant levels in excess of 10 PPM).

To facilitate these and/or other example advantages, various components of FIG. 20 can induce back pressure. For example, the permeable cover 312 can induce back pressure in the chute 1304. In such an example, the structure of the permeable cover 312 (e.g., the webbing, grille members, etc.) can inhibit the flow of oxidant out of the flow-restricted output vent 310, thereby inducing back pressure in the chute 1304. The back pressure can, additionally or alternatively, result from turbulent flow conditions and/or from a tapered or funneling design between intake and output. For instance, at least one of the chute 1304 or the flow-restricted output vent 310 is smaller than the intake vent 308. Such a reduced size of exit for oxidant (at the chute 1304 and/or the flow-restricted output vent 310) relative to the entrance size for airflow (at the intake vent 308) can induce back pressure in the chute 1304. In these or other examples, back pressure in the chute 1304 can help create a positive pressure differential within the interior volume 1400 (thereby helping to prevent the pooling of oxidant inside the interior volume 1400).

Additionally or alternatively, at least one of the manifold 1302, the chute 1304, the flow-restricted output vent 310, or the permeable cover 312 can induce turbulent flow conditions (e.g., forward of the oxidant generator coil 1310) to help promote one or more example advantages discussed above. In particular examples, the front surface 2004 can be exposed to turbulent airflow. Such turbulent airflow can include air from the fan 1300 and/or oxidant from flow recursion (e.g., oxidant that swirls or partially backflows from the chute 1304 or the permeable cover 312). In these or other examples, the combination of laminar and turbulent flow conditions (respectively before and after the oxidant generator coil 1310) can help increase utilization of the oxidant generator coil 1310 and therefore increase oxidant generation.

Any of the features, components, and/or parts, including the arrangements and configurations thereof shown in FIG. 20 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 20.

FIG. 21 illustrates an example of the oxidant generator coil cartridge 820 and the chute 1304 of the scent control apparatus 300 in accordance with one or more examples of the present disclosure. As discussed above, various components of the scent control apparatus 300 can work together to promote moisture evacuation from inside the scent control apparatus 300. In particular examples, and as shown in FIG. 21, the scent control apparatus 300 can include a gap 2100 defined (at least in part) by the oxidant generator coil cartridge 820 and the chute 1304. The gap 2100 can serve as a shunt for evacuating moisture that incidentally enters the flow-restricted output vent 310. Instead of moisture passing through the flow-restricted output vent 310 and contacting the oxidant generator coil 1310, the gap 2100 can help evacuate infiltrated moisture between the chute 1304 and the oxidant generator coil cartridge 820, thereby protecting the oxidant generator coil 1310 against moisture contact. Additionally or alternatively, the gap 2100 can help reduce or prevent the pooling of infiltrated moisture in the chute 1304 (thereby helping to ensure oxidant does not react with moisture in the chute 1304).

Any of the features, components, and/or parts, including the arrangements and configurations thereof shown in FIG. 21 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 21.

FIGS. 22-23 illustrate example first and second end portions 2200, 2300 of the oxidant generator coil cartridge 820 of the scent control apparatus 300 in accordance with one or more examples of the present disclosure. As shown, the first end portion 2200 and the second end portion 2300 opposite the first end portion 2200 suspend the oxidant generator coil 1310 to fully expose the circumferential surface of the oxidant generator coil 1310. Additionally, at least one of the first end portion 2200 or the second end portion 2300 can include keyed features to engage with the housing keys 1206. In one example, the first end portion 2200 can include protrusions 2202. In some examples, the second end portion 2300 can include recesses 2302. The protrusions 2202 and the recesses 2302 can engage the housing keys 1206 of the oxidant generator coil cartridge compartment 1204 (e.g., to ensure proper insertion of the oxidant generator coil cartridge 820 into the oxidant generator coil cartridge compartment 1204). In one or more examples, at least one of the first end portion 2200 or the second end portion 2300 can include an electrical contact 2304. The electrical contact 2304 can electrically couple with an electrical connector in the oxidant generator coil cartridge compartment 1204 (e.g., to enable the provisioning of power to the oxidant generator coil 1310).

Any of the features, components, and/or parts, including the arrangements and configurations thereof shown in FIGS. 22-23 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 22-23.

FIGS. 1-23, the corresponding text, and the examples provide several different systems, methods, techniques, components, and/or devices of a scent control system in accordance with one or more embodiments. In addition to the above description, one or more embodiments can also be described in terms of flowcharts including acts for accomplishing a particular result or performing a certain function. For example, FIG. 24 illustrates a flowchart of a series of acts 2400 for performing scent control in accordance with one or more embodiments. One or more examples of a scent control apparatus (e.g., the scent control apparatus 100, the scent control apparatus 300, etc.) may perform one or more acts of the series of acts 2400 in addition to or alternatively to one or more acts described in conjunction with other figures. While FIG. 24 illustrates acts according to one embodiment, alternative embodiments may omit, add to, reorder, and/or modify any of the acts shown in FIG. 24. The acts of FIG. 24 can be performed as part of a method. Alternatively, a non-transitory computer-readable medium can comprise instructions that, when executed by one or more processors, cause a computing device (or a computer component, such as a processor, implemented on a scent control apparatus) to perform the acts of FIG. 24. In some embodiments, a system can perform the acts of FIG. 24.

As shown, the series of acts 2400 can include an act 2402 of rotating a fan to bring air through an intake vent. In one or more examples, the act 2402 can include transmitting a signal from a processor to the power supply (or associated circuitry) to cause power to flow from the power supply to the fan. In these or other examples, the amount of power can be adjustable (e.g., to increase or decrease a fan speed or air volume throughput).

The series of acts 2400 can include an act 2404 of directing air from the fan and toward a chute aligned with the fan to directly induce contact with a rear surface of an energized oxidant generator insert (described above as the detachable oxidant generator coil 112 and the oxidant generator coil 1310). The energized oxidant generator insert can be detachable from and re-attachable to a housing of the scent control apparatus (e.g., as described above in relation to the oxidant generator coil cartridge 820). At the act 2404, turbulent flow conditions within the chute can cause additional contact between the air and a front surface of the energized oxidant generator insert opposite the rear surface (e.g., as described above in relation to FIGS. 19 and 20). In these or other examples, additional air contact with the front surface of the energized oxidant generator insert can increase oxidant output or efficiency of oxidant generation (e.g., concentration of oxidant output per joule of energy draw for energizing the fan and/or the oxidant generator insert).

The series of acts 2400 can include an act 2406 of forming oxidant based on the air contacting the energized ozone generator insert. In these or other examples, the act 2406 can include the physio-chemical steps undergone when air contacts a corona discharge generator, a corona discharge plate, an ultraviolet ozone generator, an electrolytic ozone generator, or any other type of ozone generator. Additionally or alternatively, the act 2406 can include the physio-chemical steps undergone when air contacts an ionizer (e.g., a negative ion generator) or electrostatic precipitator.

The series of acts 2400 can include an act 2408 of flowing the oxidant out through an output vent positioned below the intake vent. For example, as described above, the intake vent can be elevated relative to the output vent (e.g., to reduce perceived noise levels, enable non-mounted configurations, etc.). In one or more examples, the output vent is flow-restricted. The flow-restricted output vent can, as described above, induce the turbulent flow conditions between the energized ozone generator insert and the output vent. Additionally or alternatively, the flow-restricted output vent can induce back pressure of airflow into the front surface of the energized ozone generator insert.

The series of acts 2400 can include using any of the scent control apparatuses, scent control systems, or components disclosed herein to generate and output oxidant as disclosed herein. For example, a remote device can be used to activate, control, or terminate operation of a scent control apparatus or device as disclosed herein.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not intended to be exhaustive or to limit the embodiments to the precise forms disclosed.

It will be apparent to one of ordinary skill in the art that many modifications and variations are possible in view of the above teachings. Indeed, various inventions have been described herein with reference to certain specific aspects and examples. However, they will be recognized by those skilled in the art that many variations are possible without departing from the scope and spirit of the inventions disclosed herein. Specifically, those inventions set forth in the claims below are intended to cover all variations and modifications of the inventions disclosed without departing from the spirit of the inventions. The terms “including” or “includes” as used in the specification shall have the same meaning as the term “comprising.” Additionally, the terms “about,” “approximately,” and “substantially” should be interpreted as +/−10 percent of a given value, unless otherwise indicated.

What is claimed is:

1. A scent control apparatus, comprising:
a housing defining a first aperture and a second aperture;
an intake vent and a flow-restricted output vent respectively coinciding at least in part with the first aperture and the second aperture;
a fan positioned inside the housing and operable to bring air through the intake vent and flow air directly from the fan and into a chute leading to the flow-restricted output vent; and
a cartridge comprising an oxidant generator coil, the cartridge being removably secured to the housing to position the oxidant generator coil between the fan and the chute.

2. The scent control apparatus of claim 1, further comprising a permeable cover positioned over the flow-restricted output vent, the permeable cover configured to induce backpressure in the chute.

3. The scent control apparatus of claim 2, wherein at least one of the chute, the flow-restricted output vent, or the permeable cover is configured to induce turbulent flow conditions.

4. The scent control apparatus of claim 1, wherein a surface of the oxidant generator coil closest to the flow-restricted output vent is positioned and configured to be exposed to turbulent airflow.

5. The scent control apparatus of claim 1, wherein at least one of the chute or the flow-restricted output vent is smaller than the intake vent.

6. The scent control apparatus of claim 1, wherein the intake vent is elevated relative to the flow-restricted output vent.

7. The scent control apparatus of claim 1, wherein the intake vent and the flow-restricted output vent are both forward-facing.

8. The scent control apparatus of claim 1, wherein the fan and the oxidant generator coil are aligned relative to each other such that the air exiting the fan proceeds directly in an approximately straight flow path to the oxidant generator coil.

9. The scent control apparatus of claim 1, wherein the housing defines at least one of an exterior water-shedding feature or an internal moisture evacuation feature.

10. The scent control apparatus of claim 1, further comprising a control interface disposed on a bottom surface of the scent control apparatus.

11. A scent control system, comprising:
a scent control apparatus, comprising:
a housing defining housing openings;
an upper vent and a lower vent respectively coinciding at least in part with the housing openings and fluidly connected via a fluid flow path, the fluid flow path being constricted in size relative to the upper vent and at a location inside the housing or at the lower vent;
a fan configured to intake air through the upper vent and force the air along a direct flow path toward the lower vent;
a detachable oxidant generator coil secured to the housing and positioned in the direct flow path;
a power supply operable to energize the fan and the detachable oxidant generator coil; and
a controller configured to control operation of the scent control apparatus, the controller being communicatively coupled to at least one of the power supply or the fan; and
a remote device wirelessly coupled to the controller.

12. The scent control system of claim 11, wherein the remote device comprises a fob.

13. The scent control system of claim 11, wherein the remote device comprises a client device with a graphical user interface that, in response to user input to the graphical user interface, causes the client device to send a signal to the controller.

14. The scent control system of claim 13, wherein the graphical user interface comprises a visual indicator representative of a status of the detachable oxidant generator coil.

15. The scent control system of claim 11, wherein the controller comprises:
a processor; and
a memory device storing computer-executable instructions that, when executed by the processor, cause the processor to transmit a signal to the power supply or the fan.

16. The scent control system of claim 15, wherein the signal causes at least one of:
the power supply to change a voltage supplied to the detachable oxidant generator coil; or
the fan to change a fan speed.

17. The scent control system of claim 11, wherein the scent control apparatus is configured to be suspended via a mounting apparatus or lay on top of a support surface.

18. A scent control method performable by a scent control apparatus, the scent control method comprising:
rotating a fan to bring air through an intake vent;
directing the air from the fan and toward a chute aligned with the fan to directly induce contact with at least a rear surface of an energized oxidant generator coil of an oxidant generator insert, wherein turbulent flow conditions within the chute cause additional contact between the air and a front surface of the energized oxidant generator insert opposite the rear surface;
forming oxidant based on the air contacting the energized oxidant generator coil; and
flowing the oxidant out through an output vent positioned below the intake vent.

19. The scent control method of claim 18, wherein the oxidant generator insert is detachable from and re-attachable to a housing of the scent control apparatus.

20. The scent control method of claim 18, wherein the output vent is flow-restricted to:
induce the turbulent flow conditions between the energized oxidant generator coil and the output vent; and
induce back pressure of airflow into the front surface of the energized oxidant generator coil.
